(19) United States
(12) Patent Application Publication
(10) Pub. No.: US 2020/0128723 A1
(43) Pub. Date: Apr. 30, 2020

(54) SOIL SENSING CONTROL DEVICES, SYSTEMS, AND ASSOCIATED METHODS

(71) Applicant: Ag Leader Technology, Ames, IA (US)

(72) Inventor: Scott Eichhorn, Ames, IA (US)

(21) Appl. No.: 16/670,692

(22) Filed: Oct. 31, 2019

Related U.S. Application Data
(60) Provisional application No. 62/753,584, filed on Oct. 31, 2018.

(51) Int. Cl. A01C 5/06; G01N 33/24; A01C 7/20; A01B 79/00; G06F 16/20

(52) U.S. Cl. CPC A01C 5/064; G01N 33/24; G01N 2033/245; A01B 79/005; G06F 16/20; A01C 7/203

(57) ABSTRACT

The disclosed devices, systems, and methods relative to devices, systems and method for active control of ground engaging elements based on soil conditions. Various implementations utilize one or more sensor inputs and/or database or map inputs to adjust gauge or closing wheel down force applied by a row unit to account for the moisture of the soil being planted in real time.

### CROSS-REFERENCE TO RELATED APPLICATION(S)

This application claims the benefit under 35 U.S.C. § 119(e) to U.S. Provisional Application 62/753,584, filed Oct. 31, 2019, and entitled “Soil Sensing Control Devices, Systems, and Associated Methods, which is hereby incorporated herein by reference in its entirety for all purposes.

### TECHNICAL FIELD

The disclosure relates to devices, systems and methods for use in planting, and in particular to devices, systems, and methods for active control of ground engaging elements on row units in planting implementations. The disclosure has implications for planting of corn, soybeans, and other agricultural crops.

### BACKGROUND

In crops, such as corn and soybeans, proper seed planting depth is required to maximize crop yield. Often the weight of the planter row unit is insufficient to maintain proper planting depth in heavy and/or compacted soils. As such, many modern planting systems apply supplemental down force, as needed, to maintain proper planting depth. In some systems, gauge wheels ride on the soil surface and set the maximum planting depth and the load carried on the gauge wheels can be monitored. If the gauge wheels are not carrying enough load, the seeds likely will not be planted at the proper depth. If the gauge wheels are carrying excessive load the soil underneath the gauge wheels may compact. Compacted soil can be difficult for plant roots to penetrate and therefore may inhibit proper root development, negatively impacting yield. Soil with a high moisture content has an increased likelihood of detrimental compaction. Also, some soil types are more prone to compaction than others.

Further the amount of pressure on closing wheels may impact soil compaction and thereby yield. Various existing closing wheels have an adjustable spring to apply pressure to the closing wheels and assist in forcing the seed trench closed. Some planters have hydraulic and/or pneumatic cylinders, in place of or in addition to springs, to apply pressure to the closing wheels. Closing wheel pressure must be sufficient to provide good seed-to-soil contact when closing the furrow. However, excessive pressure may overly compact the soil around the seed, interfering with optimal plant development. Both closed and open loop control systems for closing wheels are described in U.S. Pat. No. 8,910,582, which is hereby incorporated by reference in its entirety. The control systems monitor the closing wheel pressure and adjust the applied load, but do not make adjustments for soil properties.

There is a need in the art for a control system that monitors soil properties to optimize planting unit operations, specifically gauge and closing wheel loading, to maximize crop yield.

### BRIEF SUMMARY

Discussed herein are various devices, systems, and methods for sensing soil conditions in connection with agricultural planting. In some implementations, the sensors provide inputs used to adjust the supplemental down force of various parts on a row unit.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

One Example includes a row unit down force system including a down force actuator in operational communication with a soil engaging element and constructed and arranged to apply supplemental down force to the soil engaging element; a monitoring system including at least one soil property sensor constructed and arranged to generate soil property values as sensor inputs; and a control system module, where the control system module is constructed and arranged to generate actuator command signals in response to the soil property values and sensor inputs. Implementations of Example 1 may include corresponding computer systems, apparatus, and computer programs recorded on one or more computer storage devices, each configured to perform the actions of the methods.

Implementations of this Example may also include one or more of the following features.

The row unit down force system of Example 1 where the soil engaging element is a gauge wheel.

The row unit down force system of Example 1 further including a gauge wheel load sensor in operational communication with the control system module.

The row unit down force system of Example 1 where the actuator command signals are transmitted to and control operation of the actuator.

The row unit down force system of Example 1 where the control system module utilizes the gauge wheel load and the soil property values to modify supplemental down force applied to the ground engaged element by the actuator.

The row unit down force system of Example 1 where the soil property values include at least one of soil moisture, soil pH, amount of crop residue in the soil, soil quality, soil compaction, soil nitrate level, and soil density. Implementations of the described Examples may include hardware, a method or process, or computer software on a computer-accessible medium.

Another Example includes a system for active control of at least one soil engaging element of a row unit via an actuator including a load controlling system including a soil property sensor constructed and arranged to generate soil property values. Other embodiments of this Example include corresponding computer systems, apparatus, and computer programs recorded on one or more computer storage devices, each configured to perform the actions of the methods.

Implementations of Example 2 may include one or more of the following features.

The system of Example 2 where the at least one soil engaging element is selected from at least one of a gauge wheel, a closing wheels, an opening disk, a seed firmer, and a row cleaner.

The system of Example 2 where the soil property sensor is located on the row unit.

The system of Example 2 where the soil property values include at least one of a soil moisture, a soil pH, an amount of crop residue in the soil, a soil quality, a soil compaction, a soil nitrate level, and a soil density.

The system of Example 2 where the load controlling system is constructed and arranged to generate an actuator command signal corresponding to the soil property values.

The system of Example 2 where the actuator command signal modifies down force applied by the actuator to the at least one soil engaging element.

The system of Example 2 where the soil property sensor provides soil property values prior to a seed trench being opened.

The system of Example 2 where the soil property sensor provides soil property values within an opened seed trench. Implementations of the described Examples may include hardware, a method or process, or computer software on a computer-accessible medium.

Another Example includes an active load controlling system including at least one ground engaging element, at least one soil property sensor for detecting soil property values, a control module, at least one actuator, where the control module is constructed and arranged to modify load on the ground engaging element via actuator command signals in response to soil property values. Other embodiments of this Example include corresponding computer systems, apparatus, and computer programs recorded on one or more computer storage devices, each configured to perform the actions of the methods.

Implementations may include one or more of the following features.

The active load controlling system of Example 3 where the at least one ground engaging element is a closing wheel.

The active load controlling system of Example 3 further including a closing wheel load sensor.

The active load controlling system of Example 3 where soil property values are sensed by at least one of electrical conductivity, optical spectroscopy, force penetrometers, ground penetrating radar, ultrasound, force required for tillage implement to break the soil, and interpolated soil property sensors inserted in or around the field.

The active load controlling system of Example 3 further including a database to predict soil properties values.

The active load controlling system of Example 3 where the database includes at least one of pre-recorded soil survey maps, local rainfall data, weather station history, and models of soil drainage. Implementations of the described Examples may include hardware, a method or process, or computer software on a computer-accessible medium. While multiple implementations are disclosed, still other implementations of the disclosure will become apparent to those skilled in the art from the following detailed description, which shows and describes various implementations of the invention. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 is a perspective view of a planter, according to one implementation.

FIG. 2 is a side view of a row unit, according to one implementation.

FIG. 3 is a flow chart depicting the system signal inputs, according to one implementation.

FIG. 4A is a flow chart depicting the system including GPS data, according to one implementation.

FIG. 4B is a side view of a row unit of FIG. 4A.

FIG. 4C is a flow chart depicting gauge wheel down force adjustments, according to one implementation.

FIG. 5A is a flow chart depicting the system including vehicle GPS data, according to one implementation.

FIG. 5B is a side view of a row unit of FIG. 5A.

FIG. 5C is a side view of a tillage implement in operational communication with the implementations of FIGS. 5A-5B.

FIG. 6A is a flow chart depicting the system including camera data, according to one implementation.

FIG. 6B is a side view of a row unit of FIG. 6A.

FIG. 7A is a flow chart depicting the system including several databases and precipitation records, according to one implementation.

FIG. 7B is a side view of a row unit of FIG. 7A.

FIG. 8A is a flow chart depicting the system including soil compaction sensor data, according to one implementation.

FIG. 8B is a side view of a row unit of FIG. 8A.

FIG. 9 is a side view of a row unit for closing wheel down force, according to one implementation.

FIG. 10A is a flow chart depicting the system constructed and arranged for closing wheel down force application, according to one implementation.

FIG. 10B is a side view of a row unit of FIG. 10A.

### DETAILED DESCRIPTION

The various embodiments and implementations disclosed and contemplated herein relate to devices, methods and systems for active control of one or more ground engaging elements of a planter. In various implementations, the active control of the one or more ground engaging elements may be modulated by sensor inputs, including information from soil property sensors provided to the system as sensor signals for processing.

Such ground engaging elements may include gauge wheels, closing wheels, opening disks, seed firmers, row cleaners and other elements of a row unit and/or planter as would be known to those of skill in the art. In various implementations, the applied down force is modified on the basis of soil moisture or compaction data, as well as other measured and stored data.

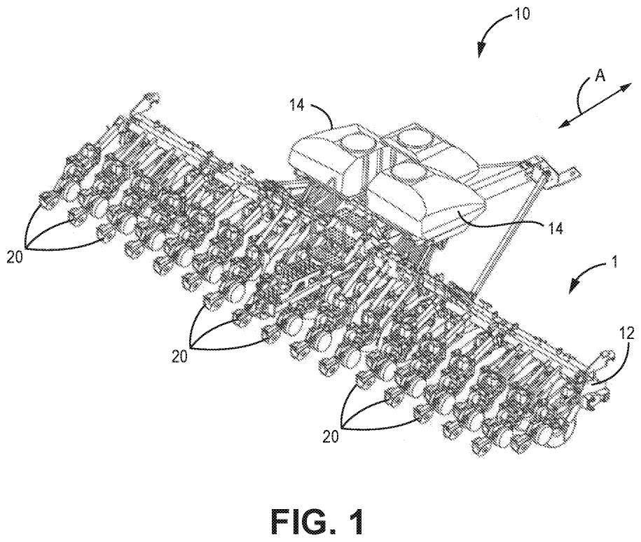

Turning to the disclosed implementations in greater detail, FIG. 1 depicts an exemplary planter 1 or seeding machine that, according to one implementation, has a soil sensing system 10. The planter 1 in this implementation is a row crop planter 1 having a planter tool bar 12 and multiple planting row units 20 mounted to the tool bar 12, as would be readily appreciated. Further implementations feature alternate configurations. In various implementations, and as described herein, the system 10 senses soil conditions in connection with a planter 1 and variably adjusts supplemental down force applied by the row units 20.

In these implementations, at least one hopper 14 is disposed on the planter 1 to hold seed, as would be understood. In further implementations, each individual row unit 20 includes at least one hopper 14 for storing and dispensing seed and/or liquids such as pesticides, herbicides and/or fertilizer and the like. It is further understood that, generally, the row units 20 on a particular planter are typically identical or substantially similar. The planter 1 moves forward and backward via the fore-aft direction shown by the reference arrow A.

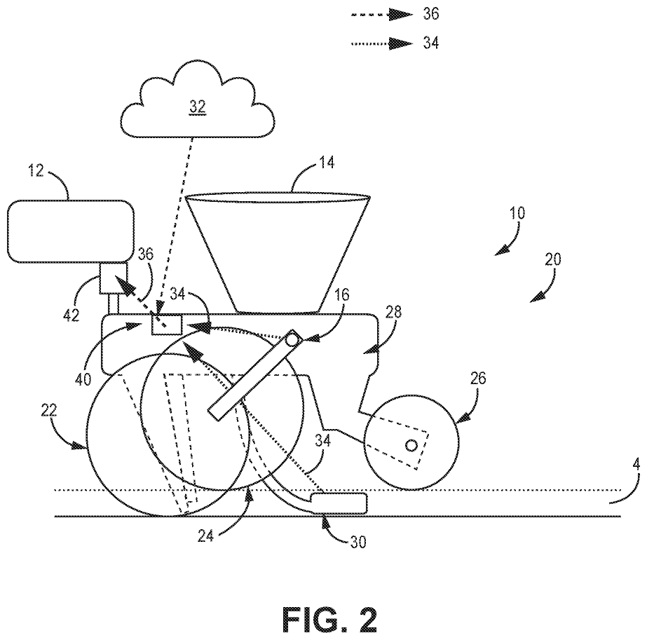

In some implementations, a row unit 20 includes a system 10 constructed and arranged for controlling gauge wheel load, as shown in FIG. 2. The row unit 20 is coupled to the tool bar 12. In various implementations the row units 20 include various devices and systems in a plurality of configurations. Row units 20 may include a hopper 14 or hoppers 14, opening disks 22, gauge wheels 24, a closing wheel 26 or wheels 26, and other components as would be generally understood in the art and that have been previously described.

Various row units 20 may optionally include one or more sensors, such as a gauge wheel load sensor 16 and/or a closing wheel sensor 18 or other sensors known in the art. Any other known components or features may be incorporated into the row units 20. It is understood that the system 10, according to any implementation disclosed and/or contemplated herein, can be incorporated into any row unit 20 having any configuration and/or sensor arrangement.

In some implementations such as that in FIG. 2, the system 10 is disposed on the row unit 20. In these implementations, a soil property sensor 30 is coupled to the row unit 20, and in the implementation of FIG. 2, more specifically to the row unit frame 28. Other arrangements are of course possible.

It is understood that maximizing crop yield requires optimizing gauge wheel load so as to balance both maintaining an optimum planting depth and preventing detrimental soil compaction. The optimum gauge wheel load may therefore vary with the soil moisture content and soil type and can be improved via the measurement via the soil property sensor 30. Additionally, the optimum gauge wheel load may vary across a field as soil moisture content and soil type changes.

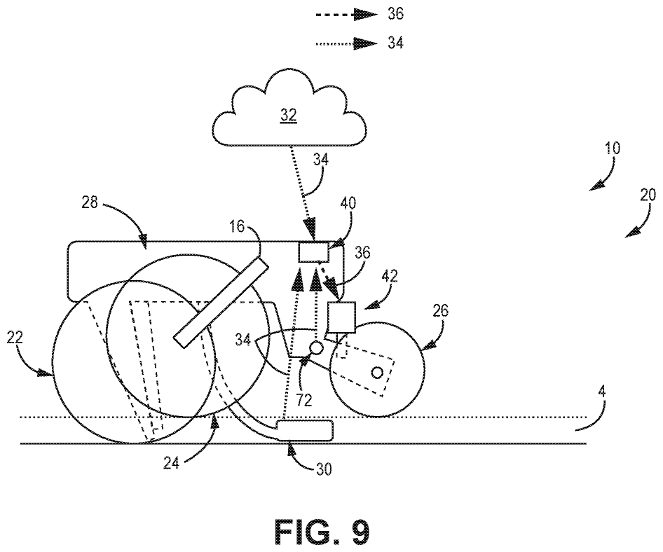

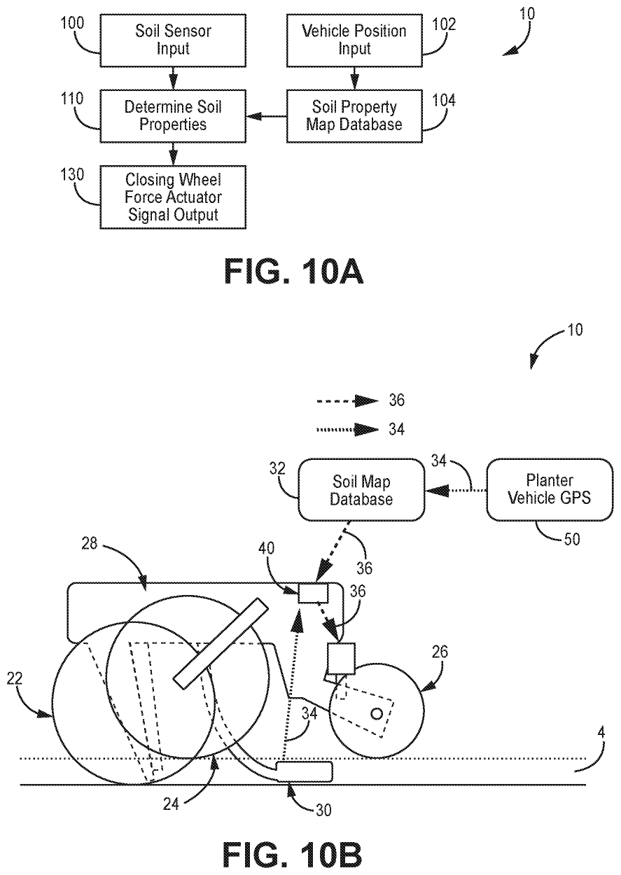

In addition to gauge wheel 24 load, other planter row unit 20 operations can be optimized using soil properties. For example, trench closing wheels also engage the soil and the closing wheel control system can be optimized by using soil property information, as described in relation to FIGS. 9-10B.

Some implementations disclosed herein relate to technologies for achieving the optimal operating conditions for the gauge wheels or other soil engaging elements in order to balance both maintaining optimal planting depth and minimizing undesirable soil compaction.

Accordingly, the soil property sensor 30 according to various implementations may sense various soil properties including but not limited to soil moisture, soil modulus, soil pH, quantity of crop residue present in the soil, soil quality, soil compaction, soil nitrate level, soil density and any other properties as would be recognized by those of skill in the art.

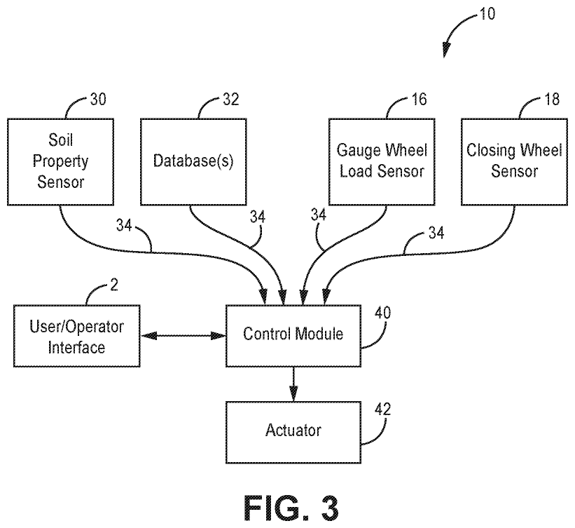

As shown in the various implementations of FIGS. 3-10B and in other various implementations, soil properties may be sensed by electrical conductivity, capacitance, video cameras, optical spectroscopy, force penetrometers, pre-recorded soil survey maps, ground penetrating radar, ultrasound, force required for tillage implement to break the soil, local rainfall data, weather station history, interpolated soil property sensors inserted in or around the field, models of soil drainage and/or by any other method known to those of skill in the art.

Soil structure is composed of mineral components, water, air, and organic materials. This structure determines how cohesive the soil is. Cohesive soils with high moisture content are more prone to compaction due to planter row unit traffic than cohesive soils with low moisture content or granular soils of any moisture content.

Knowing how prone the soil is to compaction can be used as an input to the gauge wheel control system and will affect how much load is applied to the gauge wheels, as described below.

Returning to the implementation of FIG. 2, the soil property sensor 30 may be mounted on the row unit 20 to provide sensor data before opening the seed trench 4, in the open seed trench 4, and/or after closing the seed trench 4. In addition to a soil property sensor 30, soil property data may be obtained from other devices and systems located around a field, on other vehicles or implements in or around the field, and/or from any other relevant source.

As shown in FIG. 2 and FIG. 3, in certain implementations, soil property data may be obtained from existing data relating to predicting soil properties. In one example, the system 10 can draw from established or third-party databases 32, including for example the use of pre-generated maps generated or otherwise provided by the USDA, such as a soil survey map, or other such map as would be known to those of skill in the art.

In various implementations, and as shown in FIG. 3, the data from various sources may be combined to estimate and predict soil properties via the system 10 and/or database 32. By way of example, in one such implementation, data from a soil survey map may be combined with recent rainfall data to predict soil moisture content and provide sensor input signals 34 to the control system module 40. In certain additional implementations, soil property data may also be combined with information on drain tiles in the area, which may affect soil moisture content. Myriad additional examples are of course possible.

As shown in the implementations of FIGS. 2-3, estimations of soil properties can be sent as sensor input signals 34 to the control system module 40. It is understood that the control system module 40 according to these implementations is constructed and arranged to be capable of receiving data inputs for processing and/or storage, so as to allow for the calculation of command signals 36 to be communicated to the various actuators 42 described herein and facilitate the application of down force via the soil engaging devices, such as gauge wheels 24 and/or closing wheels 26. It is further appreciated that the amount of down force to be applied by the actuators 42 is adjustable over time, in real time, and that the module 40 may perform such processing in advance of the implement applying the down force.

As shown in FIG. 3, in various implementations, the soil property sensor 30 is constructed and arranged to transmit sensor input signals 34 to the control system module 40. Input signals may be sent via wired and/or wireless mechanisms. The control system module 40 may be disposed on the row unit frame 28, as in the implementation of FIG. 2, or in any other suitable location including but not limited to the toolbar 12, planter 1, and database 32. Additionally, the gauge wheel load sensor 16 may send a sensor input signal 34 to the control system module 40.

Returning to FIG. 3, the control system module 40 receives the sensor input signals 34 and then sends—either manually or automatically—a corresponding command signal 36 to the actuator 42. In certain implementations, the actuator 42 is a single-action actuator 42. In various alternative implementations, the actuator is a double-action actuator 42. The actuator 42 may be hydraulic, pneumatic or actuated by any of the other various known methods.

In various implementations, the actuator 42 is constructed and arranged to control the supplemental downforce applied to the row unit 20. In some implementations, the supplemental down force may be applied specifically to the gauge wheel 24 and/or closing wheel 26. Various alternative implementations and configurations are possible.

Continuing with the implementations of FIGS. 2-4C, the system 10 controls gauge wheel 24 load via applied supplemental downforce such as by controlling the gauge wheel margin, average gauge wheel load, ground contact percentage, maximum gauge wheel load, minimum gauge wheel load, standard deviation of gauge wheel load, and/or any combination thereof. Controlling of the gauge wheel margin is described in further detail in U.S. Pat. No. 9,173,339, which is hereby incorporated by reference in its entirety. The system 10 after obtaining the sensor input signals 34 may adjust various parameters listed above to optimize gauge wheel 24 load or other parameters to control planting operations.

For example, soil property input signals 34 may be received by the control module 40 which may send an output command signal 36 to automatically adjust—increase or decrease—the supplemental downforce applied to the row unit 20 or gauge wheel 24, as shown in FIG. 3. The adjustments to the supplemental down force made by the system may be in addition to adjustments commanded by the gauge wheel control system. In a further implementation, the system 10 control module is in operational communication with a user/operator interface 2 configured to display sensor input signals 34 and their associated values and/or other command information to an operator via a user/operator interface 2. The operator may then issue a command to manually adjust the supplemental downforce, as would be readily appreciated.

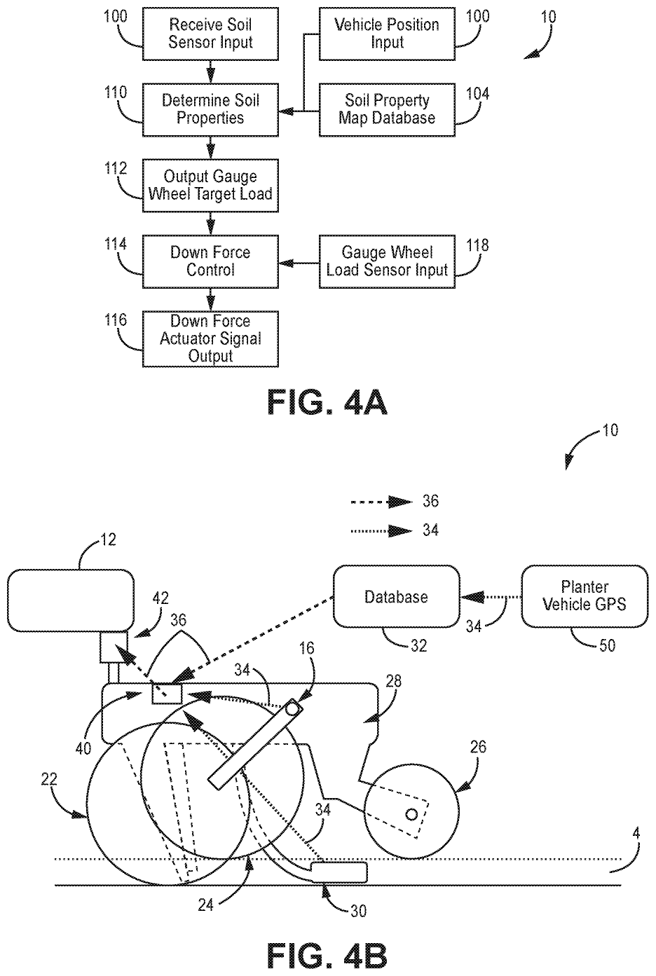

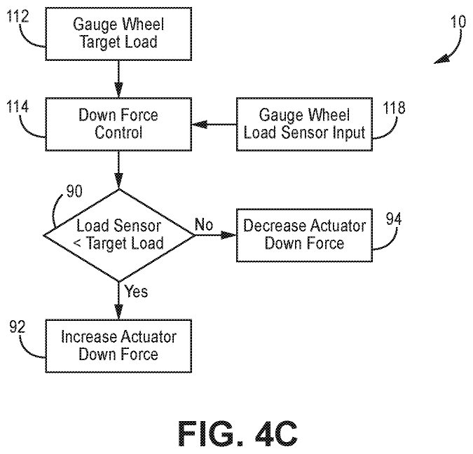

FIGS. 4A-10B depict various implementations of the system 10 utilizing a variety of sensor and database inputs processed through a variety of optional steps and sub-steps. Certain aspects of these implementations can be combined with other aspects of alternate implementations, as would be readily appreciated to adjust gauge wheel or closing wheel force.

In one example system 10 depicted in the implementation of FIGS. 4A-4C, a soil property sensor 30 such as a capacitive sensor is used to measure soil moisture content, which is received by then by the system for processing as soil sensor input (box 100).

In the implementations of FIGS. 4A-4C, a system 10 is provided wherein soil property input signals 34 may be received by the control module 40 as part of the received soil sensor input (box 100). In this implementation, a location-oriented soil property sensor is utilized, namely a vehicle-mounted GPS system 50 constructed and arranged to measure the implement position in real-time is also provided as sensor input 34 into the system 10 as vehicle position input (shown in FIG. 4A at box 102). This position data can be routed to the control module 40 for example via signal transmission 34 for processing and/or use in conjunction with data drawn from a database 32 containing, for example, USDA soil survey map data (box 104) to determine or otherwise establish the soil classification/properties (box 110) at the specified location in the field and then transmitted 36 to the module 40.

As shown in FIGS. 4A-4C, the system 10 according to these implementations is thereby constructed and arranged to utilize the determined soil properties drawn from the location and map data (box 110) to establish the initial gauge wheel target load (box 112) which is used to adjust down force control (box 114).

That is, as shown in FIG. 4C, an estimate of the soil's current susceptibility to compaction is being transmitted to the down force control (box 114) as an initial target gauge wheel load (box 112) using the soil classification, measured moisture content, or other measurements about the field, such as the map database (box 104).

It is understood that the system 10 is constructed and arranged such that if the soil has low susceptibility to compaction, then a higher gauge wheel load target (box 112) is transmitted to the down force control system (box 114) to better maintain planting depth. If the soil has high susceptibility to compaction, then a lower gauge wheel load target (box 112) is transmitted to the down force control system to better maintain planting depth. If the soil has medium susceptibility to compaction no adjustment or minimal adjustment to the gauge wheel load target (box 112) is made.

It is readily appreciated that the down force control (box 114) signal may be operably communicated to the actuator for adjustment of the actuator signal output (box 116).

As is shown in FIG. 4C, it is further understood that in these and other implementations, gauge wheel load sensor input (box 118—received from the gauge wheel load sensor 16) is also communicated to the system to further adjust the output to, for example, the actuator. In these implementations, if the gauge wheel load sensor 16 provides a load value lower than the target to the system (box 90), a command is sent to increase actuator down force (box 92). Conversely, if the load sensor value is greater than the target load, actuator force is decreased (box 94). These optional processing steps relating to the determining of the gauge wheel load target (box 112) and other adjustments to the down force control (box 114) can be performed simultaneously, in real time, or in any order, as would be readily appreciated.

In certain implementations, previously collected location and soil data can be used to adjust down force. That is, data collected previously by another vehicle, such as a tillage tractor, can be communicated to the system 10 so as to adjust the applied down force as the planter traverses the field.

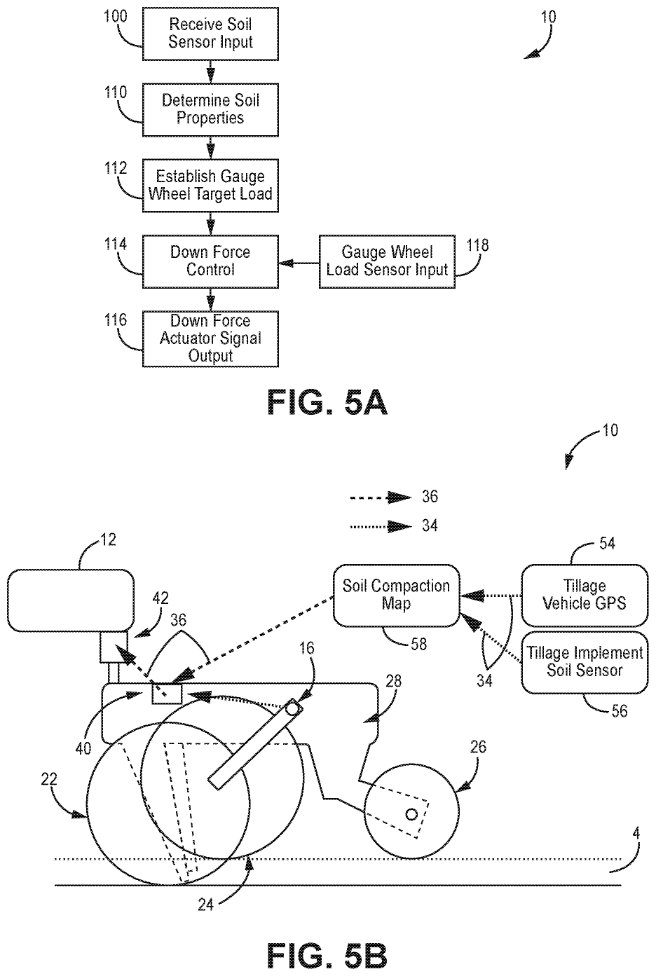

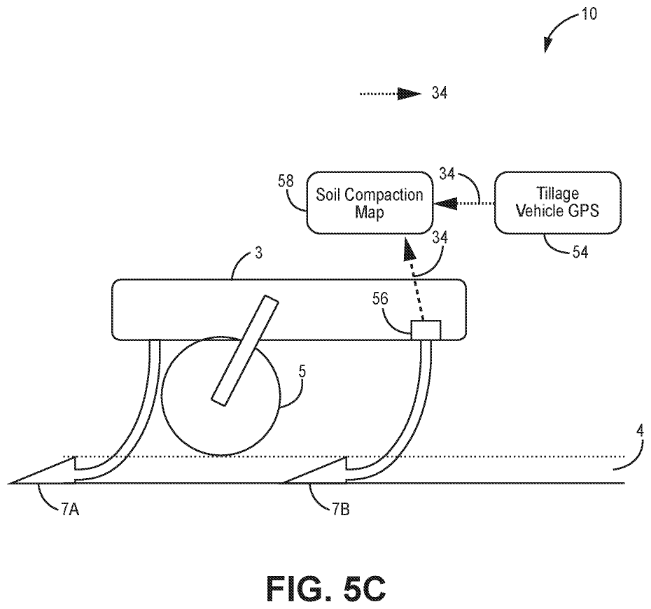

For example, as shown in FIGS. 5A-5C, the tillage tractor GPS position sensing collected from a GPS system 54 mounted on the tillage tractor may be provided as soil sensor input (box 100). This input may also include data from the tractor, such as shear force sensor 56 data 60 from tines on a tillage implement 3 that tilled the field earlier in the day are compiled to create a map 58 of field compaction conditions and determine soil properties (box 110), this sensor input signal 34 data being routed to the module 40. In one such example implementation, to measure the soil compaction susceptibility, a small section soil fractured by a leading tillage tine 7A is rolled over with a press wheel 5 mounted on the rear of the tillage implement 3 before being fractured again by a trailing tine 7B, as is shown in FIG. 5C.

In these implementations, if the shear force on this tine 7B is significantly higher than that of neighboring tines that do not trail the press wheel 5, then the soil is currently susceptible to compaction. The compaction map is then transmitted, such as wirelessly, to the GPS enabled planting implement for use with the down force control (box 114) system when it passes over the same location. It is understood that the gauge wheel load target (box 112) is processed via the down force control system (box 114) in the same manner described in the previous example.

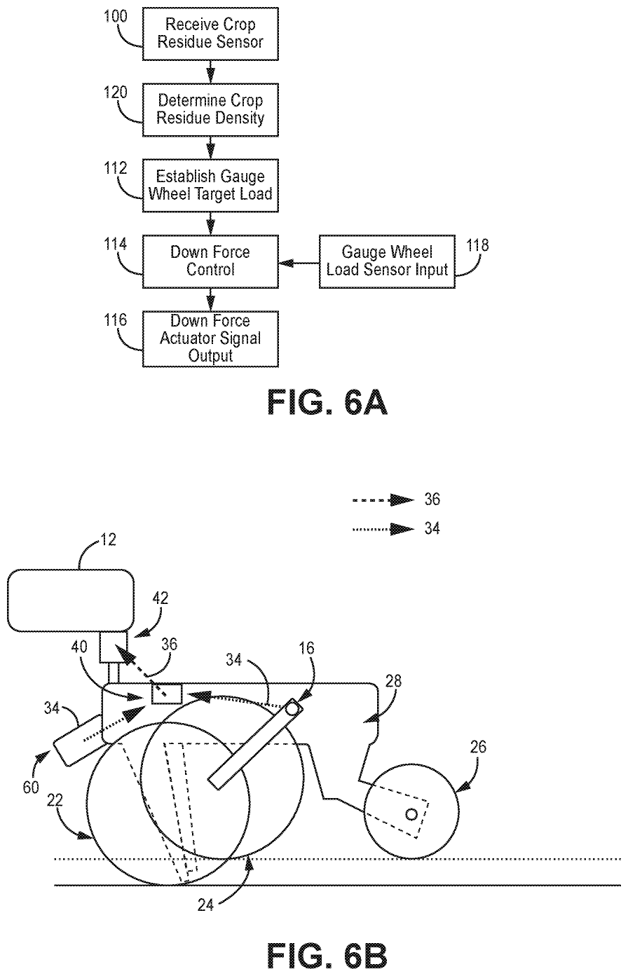

In the implementations of the system shown in FIGS. 6A-6B, a video camera 60 sensor input signal 34 is transmitted to the module 40 (in FIG. 6A at box 100) and is used to determine the degree of crop residue present (box 120) using image recognition techniques understood and appreciated in the art.

It is understood that a loss of planting depth can be caused by the gauge wheels rolling over a thick mat of crop residue. If excessive crop residue is detected a signal to increase the target gauge wheel load (box 112) is transmitted to the down force control (box 114), so as to increase the target gauge wheel load so as to further compress the crop residue and minimize planting depth loss. If crop residue is not excessive no adjustment to the gauge wheel load target is made. Additional inputs such as the gauge wheel load sensor input (box 118) can also be utilized in such implementations, as has been previously described above.

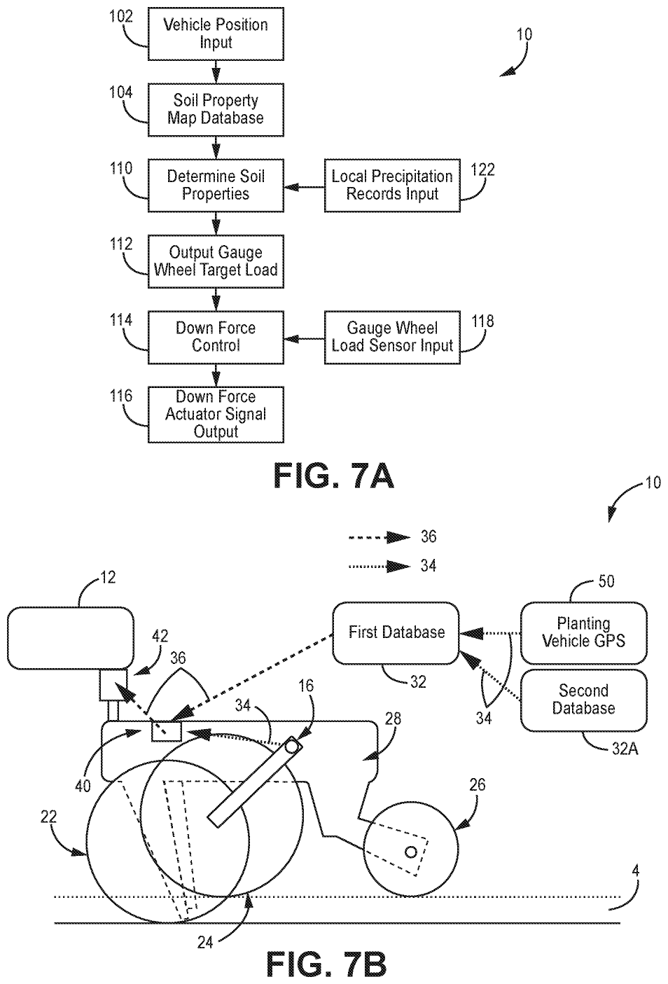

In the implementations of FIGS. 7A-7B, a vehicle-mounted GPS system 50 measures the implement's current position for transmission of a position sensor signal (box 102, via a sensor input signal 34) into the system 10. The implement's position is used in conjunction with a database 32, such as a database containing a USDA soil survey map (box 104) to determine the soil classification at that location, as has been described above. In the implementations of FIGS. 7A-7B, a second database 32A such as a database 32A storing local precipitation records transmits secondary information such as local precipitation record data (box 122) into the system 10 where the various data are synthesized with the soil map data (box 104) to generate an estimate of the soil's current susceptibility to compaction (box 110).

The susceptibility data is then transmitted to the down force control (box 114), as has been previously described. If the soil has low susceptibility to compaction, then a higher gauge wheel load target is transmitted to the down force control system to better maintain planting depth. If the soil has high susceptibility to compaction, then a lower gauge wheel load target is transmitted to the down force control system to better maintain planting depth. If the soil has medium susceptibility to compaction no adjustment to the gauge wheel load target is made, and therefore adjustment to the command signal 36 to the actuator 42.

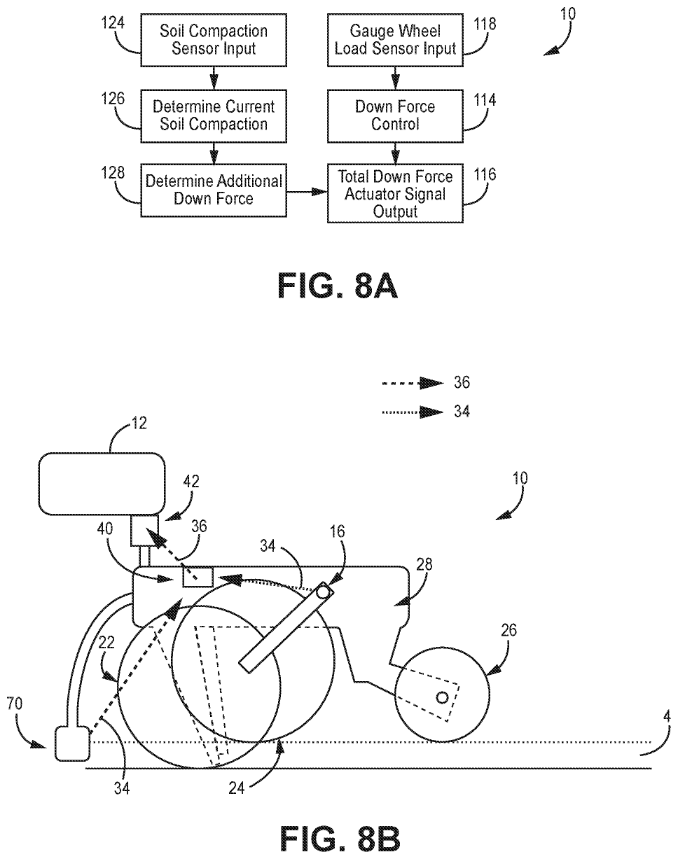

In the implementations of the system 10 shown in FIGS. 8A-8B, a soil compaction sensor 70 such as a force penetrometer 70 is mounted to the planting implement. In these implementations, the soil compaction sensor 70 is constructed and arranged to measure the pre-existing compaction of the soil in the trench 4 ahead of the opening disks 22 and gauge wheels 24.

In implementations like that of FIGS. 8A-8B, the system 10 optionally transmits sensor input 34 from the soil compaction sensor 70 to the module 40. The soil compaction data (box 124). The system processes the soil compaction data (box 124) to determine current soil compaction (box 126) and establish the amount of additional down force required (box 128), which is communicated for adjustment of the actuator signal output (box 116) to send a command signal 36 to the actuator 42.

Further, in another optional step the system 10 transmits gauge wheel sensor input 34 as a further sensor input to the module 40 for processing by the system 10 as gauge wheel load sensor input 118. As has been described above, the gauge wheel load sensor input 118 is communicated to the down force control (box 114) so as to be operably communicated to the actuator for adjustment of the actuator signal output (box 116) and therefore adjust the command signal 36 to the actuator 42.

If the system 10 determines that pre-existing compaction is high, additional force is commanded to the down force actuator to prevent planting depth loss. If pre-existing compaction is low, a reduction in force is commanded to the down force actuator to prevent detrimental soil compaction. If pre-existing compaction is nominal no adjustment to the commanded force is made.

FIG. 9 depicts another alternative implementation of the system 10 constructed and arranged for controlling closing wheel 26 supplemental down force. In these and other implementations, the system 10 may apply supplemental down force to the closing wheel 26 via a closing wheel actuator 42, as would be readily appreciated. The system 10 module may receive signal signals 34 from a database 32 or any of the other soil property sensors 30 described above in relation to FIGS. 2-8B to establish a down force actuator signal output command 36 that is relayed to the closing wheel actuator 42.

The system 10 may additionally include a control system module 40 constructed and arranged to send a command signal 36 to the closing wheel actuator 42 to adjust and/or maintain the amount of supplemental down force applied to the closing wheel. The control system module 40 may receive sensor input signals 34 from the soil property sensor 30 and the closing wheel sensor 18. The closing wheel sensor 72 could be a sensor such as the sensor described in WO2017197274A1, which is hereby incorporated by reference in its entirety.

In various implementations like that of FIG. 9, the system 10 controls the closing wheel 26 via supplemental down force by controlling at least one of the closing wheel margin, average closing wheel load, ground contact percentage, maximum closing wheel load, minimum closing wheel load, standard deviation of closing wheel load, trench closing sensor, and/or any combination thereof. Controlling the closing wheel margin is described in further detail in U.S. Pat. No. 9,173,339, which is hereby incorporated by reference in its entirety.

The implementations of FIGS. 10A-10B illustrate examples of soil properties influencing closing wheel load. In these implementations, a capacitive soil property sensor 30 is used to measure soil moisture content as sensor input 34 (box 100).

In this implementation, a vehicle-mounted GPS system 50 may be constructed and arranged to measure the implement position in real-time to the system 10 as sensor input 34 as vehicle position input (box 102). This position data (box 102) can be used for example via signal transmission 34 for processing and/or use in conjunction with data drawn from a database 32 containing, for example, USDA soil survey map data (box 104) to determine or otherwise establish the soil classification/properties (box 110) at the specified location in the field and the command transmitted 36 to the module 40 to apply closing wheel down force (box 130).

An estimate of the soil's current susceptibility to compaction is then transmitted to the closing wheel load control using the soil classification and measured moisture content. If the soil has low susceptibility to compaction, then closing wheel load is increased to provide better seed-to-soil contact. If the soil has high susceptibility to compaction, then closing wheel load is lowered to reduce detrimental compaction. If the soil has medium susceptibility to compaction no adjustment to the closing wheel load is made.

In some implementations, the system 10—after receiving the various sensor input signals 34 via the various soil property sensors 30 and/or databases 32—may adjust any of the various parameters described above, or any additional parameters as would be known, to optimize planting operations. For example, soil property input signals 34 may be used to automatically adjust—increase or decrease—the supplemental downforce applied to the closing wheel 26. The system 10 adjustment to the closing wheel 26 supplemental down force may be in addition to the adjustments indicated by a closing wheel control system. In a further implementation, the system 10 can display sensor input signals 34 and associated values as well as or other commands to an operator. The operator may then make manual adjustments to the supplemental downforce being applied to the closing wheel 26.

Although the disclosure has been described with references to various embodiments, persons skilled in the art will recognized that changes may be made in form and detail without departing from the spirit and scope of this disclosure.

What is claimed is:

1. A row unit down force system comprising:
a. a down force actuator in operational communication with a soil engaging element and constructed and arranged to apply supplemental down force to the soil engaging element;
b. a monitoring system comprising at least one sensor constructed and arranged to generate sensor inputs; and
c. a control system module,
wherein the control system module is constructed and arranged to generate actuator command signals in response to the sensor inputs.

2. The row unit down force system of claim 1, wherein the soil engaging element is a gauge wheel.

3. The row unit down force system of claim 2, further comprising a gauge wheel load sensor in operational communication with the control system module.

4. The row unit down force system of claim 3, wherein the actuator command signals are transmitted to and control operation of the actuator.

5. The row unit down force system of claim 4, wherein the control system module utilizes the gauge wheel load and the sensor inputs to modify supplemental down force applied to the ground engaged element by the actuator.

6. The row unit down force system of claim 5, wherein the sensor inputs comprise at least one of soil moisture, soil pH, amount of crop residue in the soil, soil quality, soil compaction, soil nitrate level, GPS location and soil density.

7. A system for active control of at least one soil engaging element of a row unit via an actuator comprising a load controlling system comprising a soil property sensor and constructed and arranged to generate down force command signals related to soil property values.

8. The system of claim 7, wherein the at least one soil engaging element is selected from at least one of a gauge wheel, a closing wheels, an opening disk, a seed firmer, and a row cleaner.

9. The system of claim 7, wherein the soil property sensor is located on the row unit.

10. The system of claim 7, wherein the soil property values comprise at least one of a soil moisture, a soil pH, an amount of crop residue in the soil, a GPS location, map data, database data, a soil quality, a soil compaction, a soil nitrate level, and a soil density.

11. The system of claim 7, wherein the load controlling system is constructed and arranged to generate an actuator command signal corresponding to the soil property values.

12. The system of claim 11, wherein the actuator command signal modifies down force applied by the actuator to the at least one soil engaging element.

13. The system of claim 11, wherein the soil property sensor provides soil property values prior to a seed trench being opened.

14. The system of claim 11, wherein the soil property sensor provides soil property values within an opened seed trench.

15. An active load controlling system comprising:
a. at least one ground engaging element;
b. at least one sensor for generating sensor input signals;
c. a control module constructed and arranged to process sensor input signals and generate actuator command signals;
d. at least one actuator;
wherein the control module is constructed and arranged to modify load on the ground engaging element via the actuator command signals in response to the sensor input signals.

16. The active load controlling system of claim 15, wherein the at least one ground engaging element is a closing wheel.

17. The active load controlling system of claim 16, further comprising a closing wheel load sensor.

18. The active load controlling system of claim 15, wherein at least one sensor input includes at least one of electrical conductivity, capacitance, optical spectroscopy, GPS location, camera signals, map data, force penetrometers, ground penetrating radar, ultrasound, force required for tillage implement to break the soil, and interpolated soil property sensors inserted in or around the field.

19. The active load controlling system of claim 15, further comprising a database to predict soil properties values.

20. The active load controlling system of claim 19, wherein the database includes at least one of pre-recorded soil survey maps, local rainfall data, weather station history, and models of soil drainage.
